(19) United States
(12) Patent Application Publication
(10) Pub. No.: US 2026/0253213 A1
(43) Pub. Date: Aug. 27, 2026

(54) ADVANCED IMAGE ANALYSIS SYSTEM, METHOD AND COMPUTER READABLE MEDIUM THEREOF

(71) Applicants: NATIONAL TAIWAN UNIVERSITY, Taipei City (TW); National Taiwan University Hospital, Taipei City (TW)

(72) Inventors: Wei-Chung WANG, Taipei (TW); Wei-Chih LIAO, Taipei City (TW); Po-Ting CHEN, Taipei City (TW); Da-Wei CHANG, Taipei (TW); Yen-Jia CHEN, Taipei (TW); Yan-Chen YEH, Taipei (TW)

(73) Assignees: NATIONAL TAIWAN UNIVERSITY, Taipei City (TW); National Taiwan University Hospital, Taipei City (TW)

(21) Appl. No.: 19/213,345

(22) Filed: May 20, 2025

Related U.S. Application Data
(60) Provisional application No. 63/649,801, filed on May 20, 2024.

(51) Int. Cl. G06T 7/00; A61B 6/00; A61B 6/50; G06T 3/4007; G06T 7/73; G06V 10/764; G06V 10/776; G06V 10/82; G16H 30/40

(52) U.S. Cl. CPC G06T 7/0012; A61B 6/50; A61B 6/5217; G06T 3/4007; G06T 7/73; G16H 30/40; G06T 2207/10081; G06T 2207/20081; G06T 2207/20084; G06T 2207/30056; G06T 2207/30061; G06T 2207/30096; G06V 10/764; G06V 10/776; G06V 10/82; G06V 2201/031

(57) ABSTRACT

An advanced image analysis system and an advanced image analysis method are provided. A convolutional neural network (CNN) and a transformer are used to construct a deep learning model for predictive analysis. A computed tomography (CT) image is preprocessed to obtain a preprocessed image, and the preprocessed image is analyzed by an analysis unit to determine whether an organ has a disease, so that a slice having a most important peak value, a previous slice and a next slice become key image slices, and a token and a corresponding image are displayed for medical personnel to view. As a result, whether there is the disease in the abdominal organ can be detected by using the non-contrast CT image. A computer-readable medium for executing the advanced image analysis method is further provided.

### BACKGROUND

### 1. Technical Field

The present disclosure relates to medical image judgement technology, and more particularly, to an advanced image analysis system, an advanced image analysis method and a computer-readable medium thereof.

### 2. Description of Related Art

According to research reports, pancreatic cancer (PC) is the third leading cause of cancer death in the United States. Part of the reason for the dismal prognosis of pancreatic cancer is that most patients present with unresectable or metastatic malignancies, and only a few patients can become surgical candidates and survive. According to some research reports, computed tomography (CT) scan is the main diagnostic imaging method for determining pancreatic cancer. Although tumors smaller than 2 cm have a better prognosis, about 40% of tumors are missed on CT scans. This results in post-imaging pancreatic cancers that may develop from potentially resectable lesions to unresectable metastatic tumors within a few months, thereby endangering the survival chances of pancreatic cancer patients. Therefore, effective tools are needed to help radiologists improve the limited sensitivity of small or early pancreatic cancers.

CT scans used to evaluate suspected pancreatic cancer usually require the injection or ingestion of radiographic contrast media (RCM) to enhance the difference between the lesion and its surrounding normal tissue. However, contrast-enhanced CT scans increase radiation exposure several times and may cause allergic reactions. Moreover, radiographic contrast media may also cause irreversible kidney damage in patients with chronic kidney disease (i.e., contrast-induced nephropathy).

Therefore, how to find a medical image judgment technology, especially how to judge whether an organ has a disease without using contrast agent and thereby avoiding possible problems in the use of contrast agent, has become a goal that persons skilled in the art are eager to pursue.

### SUMMARY

In order to achieve the aforementioned objective, the present disclosure provides an advanced image analysis system, which comprises: an image preprocessing module configured for locating an abdomen and a designated organ in a computed tomography (CT) image, wherein a part of the CT image whose Hounsfield unit of a stereo pixel is greater than a first setting value is regarded as a non-air area to confirm a main body area, and axial slices whose maximum component is less than a second setting value are excluded from the axial slices of the CT image to define a trunk region, thereafter an internal air ratio of each slice of the trunk region is calculated to determine an abdominal region, so that an image of the abdominal region is resampled via an interpolation, windowed and standardized to become a preprocessed image; an image judgment module configured for being connected to the image preprocessing module and having an analysis unit, wherein the analysis unit is used to extract slice features from the preprocessed image to generate a corresponding token, and determine whether the designated organ has a disease by analyzing the token corresponding to each slice and a trained classification token, wherein a slice with a greatest total attention value among the slice itself, a previous slice and a next slice is taken as a most important peak value, so that the slice itself corresponding to the most important peak value, the previous slice and the next slice become key image slices indicating that the designated organ has the disease; and an imaging module configured for being connected to the image judgment module to display the key image slices for medical personnel to view.

In an embodiment, the analysis unit further comprises: a trainable two-dimensional model for extracting the slice features from the preprocessed image to become the token; and a trainable transformer for combining the token corresponding to each slice with the classification token, and finding the most important peak value from attention value of the token corresponding to each slice after analysis by an attention mechanism, thereby obtaining the key image slices.

In an embodiment, during model training, the analysis unit inputs the token of the preprocessed image and the trainable classification token into the trainable transformer so that the trainable classification token corresponds to the token of the preprocessed image to become the classification token, and then inputs the classification token into a final linear layer to generate a logit value for binary prediction of whether or not the disease is present.

In an embodiment, after the analysis unit is trained with a training set, the logit value that leads to a greatest Youden index in a validation set is defined as a binary prediction threshold of the binary prediction.

In an embodiment, the analysis unit further establishes a stratification of different risk levels, wherein the stratification is performed by sorting all the logit values in the validation set to divide the logit values into several levels and calculating a likelihood ratio (LR) of each level, and then comparing differences in the likelihood ratios of adjacent levels, wherein two adjacent levels where a difference in the likelihood ratios between the two adjacent levels that is less than a preset value are merged to generate risk levels with clinically meaningful differences between the respective LRs.

In an embodiment, window processing and standardization of the image preprocessing module includes using a CT window of a soft tissue window, and normalizing the CT window of the soft tissue window to a range of 0-1.

In an embodiment, when the analysis unit performs model training, the model training is trained with training sets of pancreatic cancer vs. without pancreatic cancer (normal and other pancreatic diseases) and abnormal (including pancreatic cancer and other pancreatic diseases) vs. normal pancreas.

The present disclosure further discloses an advanced image analysis method, which comprises: locating, by an image preprocessing module, an abdomen and a designated organ in a computed tomography (CT) image, wherein a part of the CT image whose Hounsfield unit of a stereo pixel is greater than a first setting value is regarded as a non-air area to confirm a main body area, and axial slices whose maximum component is less than a second setting value are excluded from the axial slices of the CT image to define a trunk region; calculating, by the image preprocessing module, an internal air ratio of each slice of the trunk region to identify a lung region which will be excluded, thereby determining an abdominal region, so that an image of the abdominal region is resampled via an interpolation, windowed and standardized to become a preprocessed image; extracting, by an analysis unit of an image judgment module, slice features from the preprocessed image to generate a corresponding token, and determining whether the designated organ has a disease by analyzing the token corresponding to each slice and a trained classification token, wherein a slice with a greatest total attention value among the slice itself, a previous slice and a next slice is taken as a most important peak value, so that the slice itself corresponding to the most important peak value, the previous slice and the next slice become key image slices indicating that the designated organ has the disease; and displaying, by an imaging module, the key image slices for medical personnel to view.

In the aforementioned method, the analysis unit includes a trainable two-dimensional model and a trainable transformer, the trainable two-dimensional model extracts the slice features from the preprocessed image to become the token, and the trainable transformer combines the token corresponding to each slice with the classification token, and finds the most important peak value from attention value of the token corresponding to each slice after analysis by an attention mechanism, thereby obtaining the key image slices.

In the aforementioned method, during model training, the analysis unit inputs the token of the preprocessed image and the trainable classification token into the trainable transformer so that the trainable classification token corresponds to the token of the preprocessed image to become the classification token, and then inputs the classification token into a final linear layer to generate a logit value for binary prediction of whether or not the disease is present.

In the aforementioned method, after the analysis unit is trained with a training set, the logit value that leads to a greatest Youden index in a validation set is defined as a binary prediction threshold of the binary prediction.

In the aforementioned method, the analysis unit further establishes a stratification of different risk levels, wherein the stratification is performed by sorting all the logit values in the validation set to divide the logit values into several levels and calculating a likelihood ratio (LR) of each level, and then comparing differences in the likelihood ratios of adjacent levels, wherein two adjacent levels where a difference in the likelihood ratios between the two adjacent levels that is less than a preset value are merged to generate risk levels with clinically meaningful differences between the respective LRs.

In the aforementioned method, window processing and standardization of the image preprocessing module includes using a CT window of a soft tissue window, and normalizing the CT window of the soft tissue window to a range of 0-1.

In the aforementioned method, when the analysis unit performs model training, the model training is trained with training sets of pancreatic cancer vs. without pancreatic cancer (normal and other pancreatic diseases) and abnormal (including pancreatic cancer and other pancreatic diseases) vs. normal pancreas.

The present disclosure further discloses a computer-readable medium, applied to a computing device or a computer, which stores instructions to execute the aforementioned advanced image analysis method.

In summary, the present disclosure provides an advanced image analysis system and an advanced image analysis method. A deep learning model is constructed by combining a convolutional neural network (CNN) with a transformer to perform predictive analysis of whether the abdominal organ has a disease, wherein taking pancreatic cancer as an example, training is conducted on CT images with and without pancreatic cancer in order to predict possible pancreatic cancer and to establish the risk level of pancreatic cancer, and to display key image slices for viewing by medical personnel. In particular, the CT images are preprocessed, including determining the main body area, defining the trunk region, and performing window processing and standardization to obtain a preprocessed image. The preprocessed image is analyzed and judged by an analysis unit (i.e., a deep learning model including a trainable two-dimensional model and a trainable transformer) to determine whether there is pancreatic cancer, so that the slice with the most important peak value, the previous slice, and the next slice become key image slices. Finally, the key image slices are displayed for viewing. After verification and comparison, the present disclosure can indeed detect whether there are diseases (such as pancreatic cancer) in abdominal organs using non-contrast CT.

### BRIEF DESCRIPTION OF THE DRAWINGS

FIG. 1 is a system architecture diagram of an advanced image analysis system according to the present disclosure.

FIG. 2 is an internal architecture diagram of an image judgment module according to the present disclosure.

FIG. 3 is a step diagram of an advanced image analysis method according to the present disclosure.

FIG. 4 is a flowchart according to the present disclosure for performing computerized tomography analysis.

FIG. 5 is a flowchart of an analysis unit according to the present disclosure for performing model training.

### DETAILED DESCRIPTION

The following describes the embodiments of the present disclosure with examples. Those skilled in the art can easily understand other advantages and effects of the present disclosure from the contents disclosed in this specification. However, the present disclosure may also be implemented or applied via other different specific embodiments.

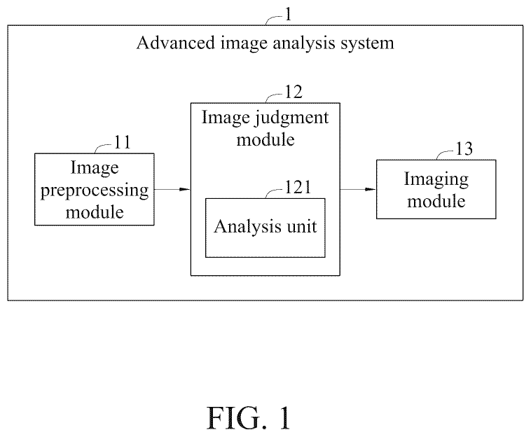

FIG. 1 is a system architecture diagram of an advanced image analysis system according to the present disclosure. Non-contrast CT is also widely used in routine health examinations and clinical examinations, but its low contrast in soft tissues hinders radiologists from detecting tumors and diagnosing other diseases, and thus needs to be improved. The present disclosure proposes a detection model for whether an organ has a disease, which can process and analyze non-contrast CT images, thereby effectively finding images that indicate a risk of an organ, which can be used by physicians for further judgment. As shown in the figure, an advanced image analysis system 1 according to the present disclosure comprises an image preprocessing module 11, an image judgment module 12, and an imaging module 13.

The image preprocessing module 11 is used to locate the abdomen and a designated organ in a CT image. Specifically, the part of the CT image whose Hounsfield unit (HU) of a stereo pixel is greater than a first setting value can be regarded as a non-air area to confirm a main body area. Then, axial slices whose maximum component is less than a second setting value are excluded from the axial slices of the CT image to define a trunk region. After that, the internal air ratio of each slice of the trunk region is calculated to determine an abdominal region. Finally, the image of the abdominal region is resampled via interpolation, windowed, and standardized to become a preprocessed image.

Specifically, the part of the CT image whose Hounsfield unit of the stereo pixel is greater than a first setting value is regarded as a non-air area. That is, the non-air area of the CT image is found, which may be a part of the patient's body. An air area is first excluded, and the main body area is further found before the next image processing. The above-mentioned judgment of whether it is an air area can be judged via the data of Hounsfield unit. The Hounsfield unit is used to designate the density of the CT. In an embodiment, the threshold (first setting value) can be set to −200 Hounsfield units as a judgment standard for judging whether it is an air area.

Afterwards, axial slices whose maximum component is less than a second setting value are excluded from the axial slices of the CT image to define a trunk region. That is, in the axial slices of the CT image, the axial slices with a larger component may be closer to the patient's trunk. On the contrary, the slices with a smaller component, such as less than the second setting value, may not be the patient's trunk, such as the limbs or the neck. In an embodiment, the second setting value can be set so that if the maximum component is less than 50%, these slices will be excluded. Based on the above, the trunk region can be defined.

Next, the internal air ratio of each slice at the trunk region is calculated to determine the abdominal region. The purpose is to find the abdominal region. The air ratio inside the body is higher, which may be close to the lungs. If the lower edge of the lungs is found, it will be helpful to define the abdominal region. In an embodiment, the slice close to the head and with an air ratio less than 20% can be identified as the lower edge of the lungs (or the upper edge of the liver), so that the abdominal region can be redefined from the trunk region.

Finally, the image of the abdominal region is resampled via interpolation. That is, each image has the same spacing, and then is windowed and standardized, wherein the spacing can be 1.5×1.5×5.0 mm, window processing converts the image into a specific width and height, and standardization is represented by 0 and 1, and finally a preprocessed image is generated. In an embodiment, the interpolation method may be trilinear interpolation or bilinear interpolation, but the present disclosure is not limited to as such.

The image judgment module 12 is connected to the image preprocessing module 11 and has an analysis unit 121. The analysis unit 121 is used to extract slice features from the preprocessed image to generate a corresponding token (also called an input sequence), and to determine whether the designated organ has a disease by analyzing the token corresponding to each slice and the trained classification token. Accordingly, a slice with the greatest total attention value among the slice itself, the previous slice and the next slice is taken as the most important peak value, so that the slice itself corresponding to the most important peak value, the previous slice and the next slice become key image slices that indicate that the designated organ is diseased. In short, the image judgment module 12 performs feature extraction on the preprocessed image to judge whether the organ has a disease, and finds out key image slices therefrom for viewing.

The analysis unit 121 extracts slice features from the preprocessed image to generate a corresponding token, and then analyzes the token corresponding to each slice and the trained classification token to determine whether the designated organ has a disease (cancer), and finds the peak value of the attention value according to the attention value of each slice. Slices with the greatest sum of attention values of the attention value of a slice corresponding to the peak value and the attention values of the previous slice and the next slice become the key image slices. The key image slices will help identify organ diseases. It should be noted that each input image (i.e., preprocessed image) can collect attention values via an attention rollout technique. Afterwards, if the attention value of a slice is higher than the attention values of the previous slice and the next slice, the attention value of the slice is regarded as the peak value, thereby finding all the peak values. After that, the peak value is compared with the sum of the attention values of the previous slice and the next slice to find the greatest one.

The imaging module 13 is connected to the image judgment module 12 to display the key image slices for medical personnel to view. In other words, the purpose of the imaging module 13 is to display the key image slices for radiologists to view and evaluate, thereby assisting the detection of organ diseases, that is, to find and display the CT slices (i.e., the key image slices with the greatest attention score in the classification results) of organs that may have abnormalities (such as a specific disease), so as to serve as a basis for physicians to further judge.

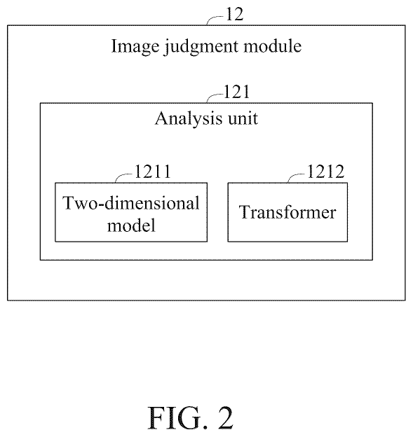

FIG. 2 is an internal architecture diagram of the image judgment module according to the present disclosure. This embodiment further illustrates the internal structure of the analysis unit 121, wherein the analysis unit 121 further comprises a two-dimensional model 1211 and a transformer 1212.

The two-dimensional model 1211 is a trainable two-dimensional model 1211, and its purpose is to extract slice features from the preprocessed image to become a token. The transformer 1212 is a trainable transformer 1212, and its purpose is to combine the token corresponding to each slice with the classification token. After analysis by the attention mechanism, the most important peak value is found from the attention value of the token corresponding to each slice, so as to obtain the key image slices.

The analysis unit 121 collects the attention weight of each input image slice from the transformer 1212 to evaluate which slices have the greatest impact on the model classification decision. For each slice, the rollout attention value between it and the classification token is calculated. When the attention value of a slice is higher than that of its two adjacent slices, it is regarded as a “peak-value slice.” Furthermore, a total attention value sum of each peak-value slice and its two adjacent slices is calculated, and the one with the greatest total attention value sum is defined as the “greatest peak value.” Finally, the slice with the greatest peak value and its two adjacent slices before and after, a total of three images, are marked as key image slices, which can be subsequently reviewed by medical personnel.

When the analysis unit 121 is trained, the token of the preprocessed image and the trainable classification token are inputted into the trainable transformer 1212, so that the trainable classification token corresponds to the token of the preprocessed image and becomes the aforementioned trained classification token, that is, to obtain the classification token of the preprocessed image. Then, the classification token is inputted into a final linear layer to generate a logit value for binary prediction of whether or not the patient has the disease. In other words, the analysis unit 121 can extract the features of each slice by including a trainable two-dimensional model 1211 (such as a trainable two-dimensional convolutional neural network [CNN]) to generate a corresponding token, that is, as one of the input sequences. Then, the tokens of all slices are inputted into the transformer 1212 together with their position codes and a classification token. Afterwards, the classification token is processed by the final linear layer to generate a logit value for binary classification.

Specifically, the classification token (CLS token) is a trainable vector that is added as a parameter at the model initialization stage and optimized end-to-end with other model weights during the entire training process. Each time the image volume (i.e., composed of multiple CT slices) is inputted, the classification token will form a set of token sequences together with the feature tokens extracted from each slice via the two-dimensional model 1211 (e.g., convolutional neural network), and further inputted into the transformer 1212 encoder. After being processed by the multi-layer self-attention mechanism, the classification token gradually integrates the global context information of all slice features and finally becomes a summary representation representing the diagnostic meaning of the entire image volume. During the training process, the output of the classification token will be sent to a linear classification layer to generate a logit value corresponding to the classification task.

In an embodiment, taking pancreatic cancer detection as an example, by minimizing the total average loss function of the binary focal loss of two classification tasks including cancer vs. non-cancer, and normal vs. abnormal, the classification token will learn how to effectively integrate information from different slices, thereby improving the overall diagnostic accuracy.

In an embodiment, after the analysis unit 121 is trained with a training set (generally, model training includes a training set, a validation set, and a reserved test set), the logit value of the validation set that results in the greatest Youden index is defined as a binary prediction threshold of the binary prediction, that is, the logit value of the one with the greatest Youden index is used as the binary prediction threshold.

In an embodiment, the analysis unit 121 also establishes a stratification of different risk levels. Specifically, the stratification is to determine the risk level of organ disease. The risk level can be accurately expressed by a likelihood ratio (LR). All the logit values in the validation set are sorted to be divided into several levels. For example, the logit values are sorted and divided into 10 levels. Then, a likelihood ratio of each level is calculated, and the differences in the likelihood ratios of adjacent levels are compared. Two adjacent levels where the difference in likelihood ratios between the two adjacent levels that is less than a preset value are merged to generate a prediction stratification that converts the logit value into a clinically meaningful risk level. Merging is to merge levels with too small differences until there are no levels that need to be merged. In this way, when testing whether an organ has a disease, it can be used as a CT slice to determine the risk of a specific disease (such as pancreatic cancer).

For example, taking pancreatic cancer detection as an example, the logit values of all samples in the validation set are first calculated and sorted in ascending order. Next, the patients predicted by the model as pancreatic cancer (PC) and non-pancreatic cancer (non-PC) are divided into ten equal parts according to their logit values, totaling 20 initial risk levels, and a corresponding likelihood ratio (LR) is calculated for each level. To ensure the discrimination between the risk levels, for adjacent levels in the same prediction group (PC or non-PC), if the LR of the latter level is less than 1.5 times the LR of the previous level, the two levels are merged. The level merging process will continue until the LR difference of all adjacent levels is greater than or equal to 1.5 times. Through the above stratification and merging strategy, the continuous logit values (that is, logit prediction values) can be converted into risk levels with clear clinical significance, thereby providing clinical personnel with pancreatic cancer risk assessment and decision assistance.

In a specific implementation, the advanced image analysis system of the present disclosure may be a hybrid deep learning system for classifying medical image data, in particular for detecting cancer in computed tomography (CT) images. The system may include a trainable two-dimensional convolutional neural network, such as a ResNet-50 architecture, to extract feature representations from individual CT image slices. These features are considered as tokens and combined with a trainable classification token to form an input sequence, which is inputted into a transformer (i.e., a transformer encoder) including multiple attention blocks, for example, four blocks, each block with eight attention heads.

Furthermore, the transformer uses trainable positional embeddings to preserve the spatial order of image slices. The classification tokens processed by the transformer can summarize the contextual information of the entire image volume and further output a corresponding logit value for classification via the final linear layer.

Taking pancreatic cancer detection as an example, the advanced image analysis system of the present disclosure can perform two binary classification tasks at the same time after training. The first task is to distinguish cancer cases from non-cancer cases (including normal and other non-cancer abnormalities), and the second task is to distinguish normal cases from all abnormal cases. The overall training loss is the average value of the binary focal loss of these two tasks. In the inference stage, the system only uses the logit value generated by the cancer vs. non-cancer classification tasks for cancer prediction and risk assessment, and uses the logit value corresponding to the maximum Youden index in the validation data set as the threshold for binary classification.

Each module of the present disclosure can be software, hardware, or firmware; if it is hardware, it can be a processing unit, a processor, a computer, or a server with data processing and computing capabilities; if it is software or firmware, it can include instructions executable by the processing unit, the processor, the computer, or the server, and can be installed in the same hardware device or distributed in different multiple hardware devices.

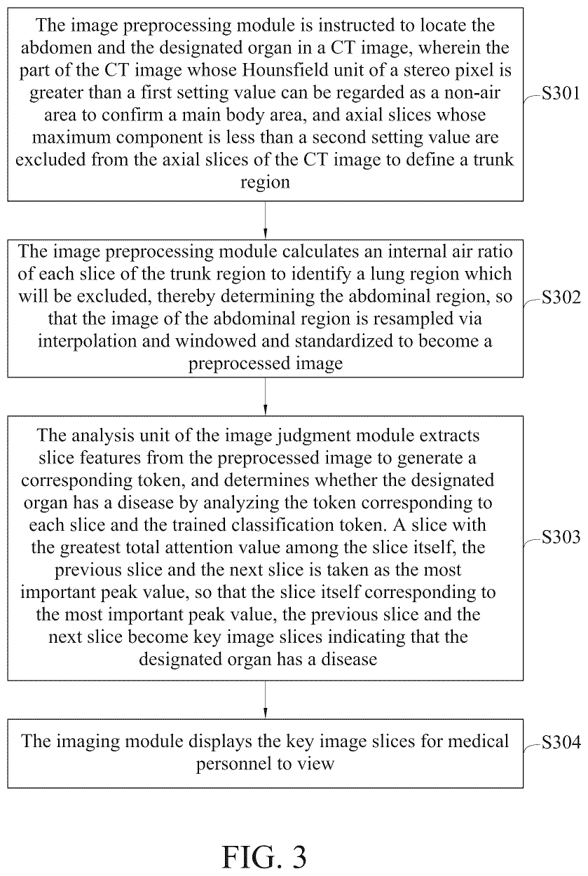

FIG. 3 is a step diagram of an advanced image analysis method according to the present disclosure.

In step S301, the image preprocessing module is instructed to locate the abdomen and the designated organ in a CT image, wherein the part of the CT image whose Hounsfield unit of a stereo pixel is greater than a first setting value can be regarded as a non-air area to confirm a main body area, and axial slices whose maximum component is less than a second setting value are excluded from the axial slices of the CT image to define a trunk region. This step describes that the image preprocessing module preprocesses the CT image, thereby locating the abdominal region and pancreatic positon. Specifically, the non-air area in the CT image is first found, so that the main body area of the patient can be confirmed. Then, the axial slices whose maximum component is less than a certain proportion are excluded, that is, the neck, limbs, etc. are excluded to define the trunk region.

In step S302, the image preprocessing module calculates an internal air ratio of each slice of the trunk region to identify a lung region which will be excluded, thereby determining the abdominal region, so that the image of the abdominal region is resampled via interpolation and windowed and standardized to become a preprocessed image. This step explains that the image preprocessing module calculates the internal air ratio of each slice at the trunk region in order to find the abdominal region. In short, when the air ratio is higher, it may be the lung range, so the air ratio less than a predetermined value is defined as the abdominal region (e.g., below the lung). After that, resampling is performed via interpolation (e.g., trilinear interpolation), that is, the spacing between each image is fixed, and then window processing (making each image the same size) and standardization are performed to become the preprocessed image.

In step S303, the analysis unit of the image judgment module extracts slice features from the preprocessed image to generate a corresponding token, and determines whether the designated organ has a disease by analyzing the token corresponding to each slice and the trained classification token. A slice with the greatest total attention value among the slice itself, the previous slice and the next slice is taken as the most important peak value, so that the slice itself corresponding to the most important peak value, the previous slice and the next slice become key image slices indicating that the designated organ has a disease. This step describes that the analysis unit of the image judgment module will extract slice features to become a token corresponding to each slice. In addition, the token corresponding to each slice and the trained classification token are analyzed to determine whether the designated organ has a disease. According to the comparison of the attention value of each slice, the peak value is found, and the sums of the peak values of each slice and its previous and next slices are compared. The greatest sum of peak values is taken as the most important peak value, and the corresponding slice and its previous and next slices become the key image slices to assist in determining organ diseases.

In an embodiment, the analysis unit includes a trainable two-dimensional model and a trainable transformer. The trainable two-dimensional model is used to extract slice features from the preprocessed image to become a token, and the trainable transformer is used to combine the token corresponding to each slice with the classification token. After analysis by the attention mechanism, the most important peak value is found from the attention value of the token corresponding to each slice to obtain the key image slices.

In step S304, the imaging module displays the key image slices for medical personnel to view. This step describes that the imaging module can mark the key image slices, that is, point out and display the key images for subsequent observation.

In an embodiment, during the model training, the analysis unit inputs the token of the preprocessed image and the trainable classification token into the trainable transformer so that the trainable classification token corresponds to the token of the preprocessed image and becomes the classification token, and then inputs the classification token into a final linear layer to generate a logit value for binary prediction. After the analysis unit is trained with the training set, the logit value that leads to the greatest Youden index in the validation set is defined as a binary prediction threshold for binary prediction.

In addition, the analysis unit can establish a stratification of different risk levels, wherein the validation of the stratification can be performed by sorting all the logit values in the validation set into several levels. Then, a likelihood ratio (LR) of each level is calculated, and the adjacent two levels whose likelihood ratios are similar or whose difference is less than a preset value are merged to generate a prediction stratification by converting the logit values into a clinically meaningful risk level, wherein the stratification indicates high or low risk when tested.

In addition, during the recognition phase, the most concerned slices (tokens) are selected from the transformer of the analysis unit and displayed as a visual interpretation. Specifically, the attention weight of each inputted image inside the transformer can be collected using the attention rollout technique. For each slice, the attention value between the slice itself and the classification token is extracted for further analysis. If the attention value of the previous and next slices is low, the slice is determined as a peak value. After all peak values are identified, the peak value with the greatest sum of attention values among the slice itself, its previous slice, and its next slice is selected as the most important peak value. Finally, the slice corresponding to the most important peak value and its previous and next slices are selected as key image slices and displayed for visual evaluation by radiologists.

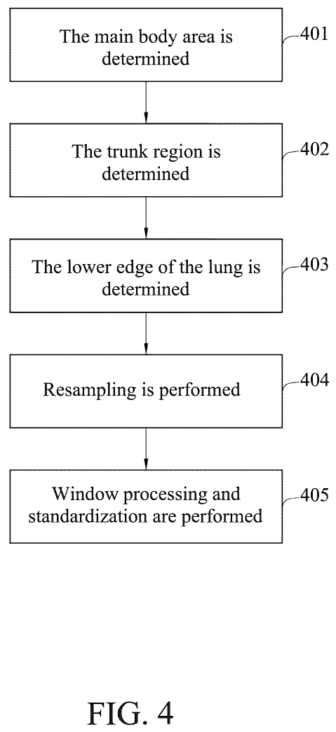

FIG. 4 is a flowchart according to the present disclosure for performing computerized tomography analysis, taking pancreatic cancer detection as an example.

In process 401, the main body area is determined. This process illustrates that a threshold processing is performed on the image according to the Hounsfield unit (HU) with a value of −200, wherein voxels with values greater than the threshold are regarded as non-air areas, and then the main body area can be determined by filling the holes in the non-air areas.

In process 402, the trunk region is determined. This process illustrates that in order to remove the areas of the limbs, neck and extremities, it is necessary to determine the largest connected component on each axial slice and remove other smaller components. Further, the slices where the largest component occupies less than 50% of the area are excluded. Then, the retained CT image volume is cropped into a smaller rectangular cuboid and precisely bound to the remaining components on each slice.

In process 403, the lower edge of the lung is determined. This process describes that in order to make the height of the cuboids between each image equal, for each slice, the proportion of air inside the body part is calculated, and the slice closest to the head and with a proportion less than 20% is determined as the lower edge of the lung (or the upper edge of the liver). For example, only the slices within 24 cm below the specific slice are retained, and other slices are removed.

In process 404, resampling is performed. This process illustrates that after cropping to the abdominal area, the image is resampled to a spacing of 1.5×1.5×5.0 mm using, for example, trilinear interpolation.

In process 405, window processing and standardization are performed. This process describes that the aforementioned image slices are windowed and normalized to 0 and 1. Specifically, a CT window (soft tissue window=width: 350 HU, level: 50 HU; level is a pancreas average intensity [HU value] of the pancreas segmentation mask generated by a pre-trained pancreas segmentation deep learning model) is used and converted into a parallel channel and normalized to the [0, 1] range.

Finally, the image volume is resized to 320×288×32 voxels by fixed center cropping or padding with zeros to become a preprocessed image. That is, the data is resized to a uniform shape of 320×288×32 voxels by center cropping or zero padding, and the center position is determined according to the pancreas segmentation mask.

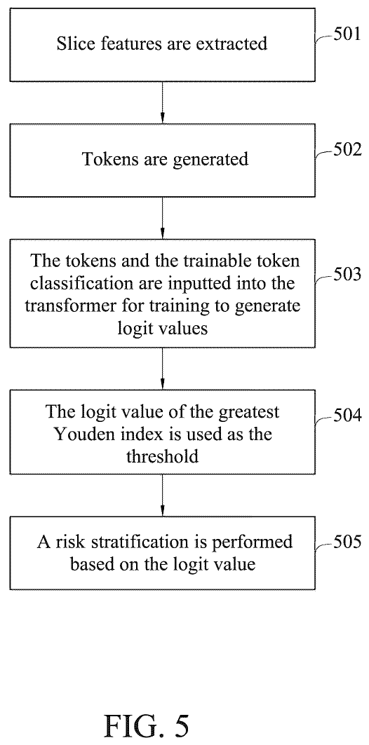

FIG. 5 is a flowchart of the analysis unit according to the present disclosure for performing model training, which illustrates how a deep learning model is trained. In short, the present disclosure uses a CNN-Transformer hybrid deep learning model to distinguish image volumes with pancreatic cancer and without pancreatic cancer, taking the identification of pancreatic cancer as an example. The architecture of the model includes a trainable two-dimensional model (2D ResNet-50 Model) and a trainable transformer. The two-dimensional model is a convolutional neural network (CNN). The transformer has 4 blocks, and each block has 8 heads.

In process 501, slice features are extracted. This process describes that each slice of the inputted preprocessed image first passes through the two-dimensional model to extract slice features.

In process 502, tokens are generated. Continuing from the previous process, after the extraction of the slice features is completed, they will become tokens.

In process 503, the tokens and the trainable token classification are inputted into the transformer for training to generate logit values. This process illustrates that the tokens from the inputted image volume slices and the trainable classification tokens are further inputted into the transformer embedded with a trainable position, so that the outputted classification tokens are inputted into a final linear layer to generate the logit values for binary prediction.

Then, the model was trained on a local training set to classify images as having cancer or not having cancer, wherein the model's training batch size was 48, which is the number of training samples captured in one training. A total of 300 training epochs were trained, and an optimizer (e.g., AdamW) is used so that the cosine-annealing learning rate is reduced from 1×10⁻⁴ to 10⁻⁶ every 75 training epochs and repeated four times.

To avoid gradient explosion during training, if the norm of the gradient is too large, the norm of the gradient is limited to 10, and mixed precision is used to speed up the training process. The training of the model involves two binary classifications at the same time, including PC (pancreatic cancer) vs. non-PC (normal and other pancreatic diseases [OPD]), and abnormal (PC and OPD) vs. normal. The loss function is defined as the mean of the focal loss of the two binary classifications. Only the logit value of PC vs. non-PC is further used to evaluate the binary classification and risk level.

In process 504, the logit value of the greatest Youden index is used as the threshold. This process illustrates that the threshold of binary prediction is selected from the logit value that leads to the greatest Youden index in the validation set.

In process 505, a risk stratification is performed based on the logit value. This process illustrates the establishment of risk stratification via the logit value.

In order to further provide the possibility of PC based on the prediction of the deep learning model, the model output is further stratified into different risk levels, where the likelihood ratio (LR) can be determined through a local validation set.

Specifically, to determine the risk level, the logit values of the deep learning model for the patients in the validation set are calculated and sorted in ascending order. The patients predicted as PC and non-PC are divided into 10 levels on average, and the LR within these 20 levels is calculated. If the LR of the latter level is less than 1.5 times that of the previous level, two adjacent levels from the same binary prediction (PC or non-PC) are merged into one level. The merging process is repeated until no levels need to be merged. If LR≤0.01 or LR>100, the diagnostic confidence of each level is “very strong”; if 0.01<LR≤0.1 or 10<LR≤100, the diagnostic confidence is “strong”; if LR>0.1 or LR≤10, the diagnostic confidence is “medium.”

The system of the present disclosure was verified. In the reserved test set, the system of the present disclosure (which can be developed into a computer-aided design [CAD] tool) can distinguish the non-contrast CT images of normal people and other pancreatic diseases (OPD), with a sensitivity of 90.8%, a specificity of 93.0% (97.3% for normal people and 83.0% for OPD), and an accuracy of 92.5%. In addition, among the 129 correctly diagnosed PCs, based on the review of the corresponding enhanced images by experienced radiologists, the key non-contrast images detected by the system of the present disclosure includes 95 (73.6 %) patients with tumors and 21 (16.3%) patients with diagnostic secondary signs of PC (such as pancreatic duct dilatation, extrahepatic bile duct dilatation, or intrahepatic bile duct dilatation). This indicates that the present disclosure can indeed detect/diagnose pancreatic cancer (PC) on the non-contrast CT images and indicate key images to facilitate physicians' review.

It should be noted that when the analysis unit of the present disclosure performs model training, it is trained with the training sets of PC vs. non-PC (normal and other pancreatic diseases [OPD]) and abnormal (PC and OPD) vs. normal mentioned above. Compared to the current model for pancreatic cancer judgment, which is only trained with a single type of data training set of pancreatic cancer and non-pancreatic cancer during the training process, the detection model in the analysis unit of the present disclosure is trained with the aforementioned training sets of different types of data, and can more accurately judge pancreatic cancer. Therefore, the detection model proposed by the present disclosure is significantly superior to the existing pancreatic cancer judgment model.

In addition, the present disclosure also discloses a computer-readable medium, which is applied to a computing device or computer having a processor (for example, central processing unit [CPU], graphics processing unit [GPU], etc.) and/or a memory, and stores instructions. Moreover, the computing device or computer can be used to execute the computer-readable medium via the processor and/or memory, so as to execute the above-mentioned method and each step process when executing the computer-readable medium. In an embodiment, the computer-readable medium is a non-transitory computer-readable storage medium.

In summary, the present disclosure proposes an advanced image analysis system, an advanced image analysis method and a computer-readable medium thereof. Via a convolutional neural network and a transformer, the present disclosure constructs a deep learning model to perform detection and analysis of whether an organ has a disease (such as pancreatic cancer), wherein the CT image is preprocessed, including determining the main body area, defining the trunk region, and performing window processing and standardization to obtain a preprocessed image. The preprocessed image is analyzed and judged by an analysis unit (i.e., a deep learning model including a trainable two-dimensional model and a trainable transformer) to confirm whether the organ has a disease. Moreover, according to the attention value of each slice, a slice having the most important peak value, the previous slice and the next slice become key image slices. Finally, the key image slices are displayed for physicians to view. Based on the above, the present disclosure can provide effective organ disease (such as pancreatic cancer) detection for non-contrast CT images.

The foregoing embodiments are provided for the purpose of illustrating the principles and effects of the present disclosure, rather than limiting the present disclosure. Anyone skilled in the art can modify and alter the above embodiments without departing from the spirit and scope of the present disclosure. Therefore, the scope of protection with regard to the present disclosure should be as defined in the accompanying claims listed below.

1. A advanced image analysis system, comprising:
an image preprocessing module configured for locating an abdomen and a designated organ in a computed tomography (CT) image, wherein a part of the CT image whose Hounsfield unit of a stereo pixel is greater than a first setting value is regarded as a non-air area to confirm a main body area, and axial slices whose maximum component is less than a second setting value are excluded from the axial slices of the CT image to define a trunk region, thereafter an internal air ratio of each slice of the trunk region is calculated to determine an abdominal region, so that an image of the abdominal region is resampled via an interpolation, windowed and standardized to become a preprocessed image;
an image judgment module configured for being connected to the image preprocessing module and having an analysis unit, wherein the analysis unit is used to extract slice features from the preprocessed image to generate a corresponding token, and determine whether the designated organ has a disease by analyzing the token corresponding to each slice and a trained classification token, wherein a slice with a greatest total attention value among the slice itself, a previous slice and a next slice is taken as a most important peak value, so that the slice itself corresponding to the most important peak value, the previous slice and the next slice become key image slices indicating that the designated organ has the disease; and
an imaging module configured for being connected to the image judgment module to display the key image slices for medical personnel to view.

2. The advanced image analysis system of claim 1, wherein the analysis unit further comprises:
a trainable two-dimensional model for extracting the slice features from the preprocessed image to become the token; and
a trainable transformer for combining the token corresponding to each slice with the classification token, and finding the most important peak value from attention value of the token corresponding to each slice after analysis by an attention mechanism, thereby obtaining the key image slices.

3. The advanced image analysis system of claim 2, wherein during model training, the analysis unit inputs the token of the preprocessed image and the trainable classification token into the trainable transformer so that the trainable classification token corresponds to the token of the preprocessed image to become the classification token, and then inputs the classification token into a final linear layer to generate a logit value for binary prediction of whether or not the disease is present.

4. The advanced image analysis system of claim 3, wherein, after the analysis unit is trained with a training set, the logit value that leads to a greatest Youden index in a validation set is defined as a binary prediction threshold of the binary prediction.

5. The advanced image analysis system of claim 4, wherein the analysis unit further establishes a stratification of different risk levels, wherein the stratification is performed by sorting all the logit values in the validation set to divide the logit values into several levels and calculating a likelihood ratio (LR) of each level, and then comparing differences in the likelihood ratios of adjacent levels, wherein two adjacent levels where a difference in the likelihood ratios between the two adjacent levels that is less than a preset value are merged to generate risk levels with clinically meaningful differences between the respective LRs.

6. The advanced image analysis system of claim 1, wherein window processing and standardization of the image preprocessing module includes using a CT window of a soft tissue window, and normalizing the CT window of the soft tissue window to a range of 0-1.

7. The advanced image analysis system of claim 1, wherein when the analysis unit performs model training, the model training is trained with training sets of pancreatic cancer vs. without pancreatic cancer (normal and other pancreatic diseases) and abnormal (including pancreatic cancer and other pancreatic diseases) vs. normal pancreas.

8. An advanced image analysis method, comprising:
locating, by an image preprocessing module, an abdomen and a designated organ in a computed tomography (CT) image, wherein a part of the CT image whose Hounsfield unit of a stereo pixel is greater than a first setting value is regarded as a non-air area to confirm a main body area, and axial slices whose maximum component is less than a second setting value are excluded from the axial slices of the CT image to define a trunk region;
calculating, by the image preprocessing module, an internal air ratio of each slice of the trunk region to identify a lung region which will be excluded, thereby determining an abdominal region, so that an image of the abdominal region is resampled via an interpolation, windowed and standardized to become a preprocessed image;
extracting, by an analysis unit of an image judgment module, slice features from the preprocessed image to generate a corresponding token, and determining whether the designated organ has a disease by analyzing the token corresponding to each slice and a trained classification token, wherein a slice with a greatest total attention value among the slice itself, a previous slice and a next slice is taken as a most important peak value, so that the slice itself corresponding to the most important peak value, the previous slice and the next slice become key image slices indicating that the designated organ has the disease; and
displaying, by an imaging module, the key image slices for medical personnel to view.

9. The advanced image analysis method of claim 8, wherein the analysis unit includes a trainable two-dimensional model and a trainable transformer, the trainable two-dimensional model extracts the slice features from the preprocessed image to become the token, and the trainable transformer combines the token corresponding to each slice with the classification token, and finds the most important peak value from attention value of the token corresponding to each slice after analysis by an attention mechanism, thereby obtaining the key image slices.

10. The advanced image analysis method of claim 9, wherein during model training, the analysis unit inputs the token of the preprocessed image and the trainable classification token into the trainable transformer so that the trainable classification token corresponds to the token of the preprocessed image to become the classification token, and then inputs the classification token into a final linear layer to generate a logit value for binary prediction of whether or not the disease is present.

11. The advanced image analysis method of claim 10, wherein, after the analysis unit is trained with a training set, the logit value that leads to a greatest Youden index in a validation set is defined as a binary prediction threshold of the binary prediction.

12. The advanced image analysis method of claim 11, wherein the analysis unit further establishes a stratification of different risk levels, wherein the stratification is performed by sorting all the logit values in the validation set to divide the logit values into several levels and calculating a likelihood ratio (LR) of each level, and then comparing differences in the likelihood ratios of adjacent levels, wherein two adjacent levels where a difference in the likelihood ratios between the two adjacent levels that is less than a preset value are merged to generate risk levels with clinically meaningful differences between the respective LRs.

13. The advanced image analysis method of claim 8, wherein window processing and standardization of the image preprocessing module includes using a CT window of a soft tissue window, and normalizing the CT window of the soft tissue window to a range of 0-1.

14. The advanced image analysis method of claim 8, wherein when the analysis unit performs model training, the model training is trained with training sets of pancreatic cancer vs. without pancreatic cancer (normal and other pancreatic diseases) and abnormal (including pancreatic cancer and other pancreatic diseases) vs. normal pancreas.

15. A computer-readable medium, applied to a computing device or a computer, which stores instructions to execute the advanced image analysis method of claim 8.
